(19) United States
(12) Patent Application Publication
(10) Pub. No.: US 2021/0196582 A1
(43) Pub. Date: Jul. 1, 2021

(54) METAL EXTRACTION SHEET, MASK PACK INCLUDING SAME AND METHOD FOR MANUFACTURING THE METAL EXTRACTION SHEET

(71) Applicant: Amorepacific Corporation, Seoul (KR)

(72) Inventors: Jeong Eun SEO, Yongin-si (KR); Hyoung June KIM, Yongin-si (KR); Il Doo KIM, Daejeon (KR); Jae Hyeong BAE, Daejeon (KR); Doo Young YOUN, Daejeon (KR)

(73) Assignee: Amorepacific Corporation, Seoul (KR)

(21) Appl. No.: 17/136,251

(22) Filed: Dec. 29, 2020

(30) Foreign Application Priority Data
Dec. 31, 2019 (KR) 10-2019-0179997

(51) Int. Cl. A61K 8/02; A61K 8/41; A61Q 19/00

(52) U.S. Cl. CPC A61K 8/0233; A61K 8/415; A61Q 19/00; A61K 8/0212; B82Y 5/00; A61K 2800/45; A61K 2800/612; A61K 2800/81; A61K 2800/10; A61K 2800/413

(57) ABSTRACT

The present disclosure relates to a metal material extraction sheet. According to an aspect of the disclosure, the metal material extraction sheet having a predetermined area may be provided, wherein dopamine whose color is changed when reacting with a metal material is provided on at least a part of the outer surface of the metal material extraction sheet.

### CROSS-REFERENCE TO RELATED APPLICATION

This application is based on and claims priority of Korean Patent Application No 10-2019-0179997, filed on Dec. 31, 2019 with the Korean Intellectual Property Office, the entire contents of which are hereby incorporated by reference.

### TECHNICAL FIELD

One aspect of the present disclosure relates to a metal material extraction sheet, a mask pack including the same, and a method of preparing a metal material extraction sheet.

### BACKGROUND

Recently, social interest in the atmosphere environment problem caused by fine dust is increasing, and the demand for related industries is also increasing accordingly.

Fine dust contains various complex components such as organic substances, sulfates, carbon substances, heavy metals, and the like, and particularly, continuous exposure of the human body to heavy metals contained in fine dust may result in heavy metal poisoning diseases. In addition, when these heavy metals are accumulated in pores or sweat glands of the skin, they may cause various skin diseases.

Since it is difficult to detect and remove heavy metals in such fine dust, various researches have recently been conducted to efficiently remove and block heavy metal components of fine dust. However, there is no product for easily and simply detecting and removing heavy metals accumulated on the skin by fine dust.

On the other hand, since a metal material is used as a raw material for cosmetics, problems such as skin trouble may occur if makeup of using a cosmetic material containing such a metal material is not erased completely from the skin. However, there is no product for easily and simply detecting and removing metal materials contained in raw materials of cosmetics.

### SUMMARY

The exemplary embodiments of the disclosure, which have been conceived to address above-described drawbacks, is to provide a metal material extraction sheet that can show the metal material to users by visualizing the metal material removed from the skin while removing metal material accumulated on the skin.

### Technical Solution

According to one aspect of the present invention, there is provided a metal material extraction sheet that has a predetermined area, and dopamine whose color changes when reacting with metal material and which is provided on at least a part of an outer surface of the metal material extraction sheet.

Further, there is provided a metal material extraction sheet, wherein the metal material extraction sheet is formed by intertwining or aligning in a predetermined direction nanofibers in which a polymer and the dopamine are mixed in a preset ratio.

Further, there is provided a metal material extraction sheet, wherein the diameter of the nanofibers is between 10 nm and 50 nm.

Further, there is provided a metal material extraction sheet, wherein the average pore size of the nanofibers is between 0.1 μm and 1.0 μm.

Further, there is provided a metal material extraction sheet, wherein the weight ratio of the dopamine and the polymer is between 1:1 and 1:10.

Further, there is provided a metal material extraction sheet, wherein the metal material extraction sheet further comprises a functional raw material, and wherein the functional raw material includes one or more of a vitamin, a whitening agent, a skin tone changing agent, an anti-wrinkle agent, an anti-aging agent, an anti-acne agent, a moisturizing agent, and a nutritional agent.

Further, there is provided a metal material extraction sheet, wherein the metal material extraction sheet is impregnated with liquid.

Further, there is provided a metal material extraction sheet, wherein the liquid is a basic material having a pH of 8.5 or higher.

Further, there is provided a metal material extraction sheet, wherein the metal material extraction sheet further includes a sheet layer having a predetermined area, and the dopamine is provided on one surface of the sheet layer to form a surface layer thereof.

Further, there is provided a metal material extraction sheet, wherein the surface layer is formed in a region of 1/10 or less of the surface area of the sheet layer.

Further, there is provided a metal material extraction sheet, wherein the surface layer is provided on both surfaces of the sheet layer.

Further, there is provided a mask pack comprising a metal material extraction sheet, wherein the metal material extraction sheet is deformable to correspond to a user's body shape, and the dopamine is provided to a user's body side of the metal extraction sheet. The term “mask pack” used herein refers to a sheet in various shapes conforming a shape of the target part of the user. For example, a facial mask pack may refer to a sheet of a facial shape or a shape suitable to put on face of the user, optionally with holes exposing eyes, nostrils, and mouth of the user. A mask pack, a sheet mask, and a mask sheet are used interchangeably.

According to another aspect of the present invention, there is provided a method of preparing a metal material extraction sheet, the method comprising: preparing a spinning solution containing dopamine; electrospinning the spinning solution to form nanofibers; and laminating the nanofibers to form the metal material extraction sheet.

The metal material extraction sheet according to the embodiments of the present disclosure has the advantage of removing the metal material accumulated on the skin and show the metal material removed from the skin to users by visualizing it.

### BRIEF DESCRIPTION OF THE DRAWINGS

FIG. 1 is a schematic view representing a metal material extraction sheet according to an embodiment of the present disclosure.

FIG. 2 is a diagram conceptually representing a process in which dopamine in a surface layer of FIG. 1 reacts with a metal material, so that the dopamine is polymerized into polydopamine.

FIG. 3 schematically represents a solution for preparing the surface layer of FIG. 1.

FIG. 4 schematically represents an electrospinning apparatus for preparing the surface layer of a metal material extraction sheet of FIG. 1 prepared by the electrospinning apparatus.

FIG. 5 represents a mask pack to which the metal material extraction sheet of FIG. 1 is applied.

FIG. 6 represents an embodiment of the invention where the metal material extraction sheet of FIG. 1 is applied to a part of a patch.

FIG. 7 represents an embodiment of the invention where the metal material extraction sheet of FIG. 1 is applied to a cotton pad.

FIG. 8 is a schematic view representing a metal material extraction sheet according to another embodiment of the present disclosure.

### DETAILED DESCRIPTION

Hereinafter, specific exemplary embodiments of this disclosure will be described in detail with reference to the drawings.

Additionally, it is noted that in the description of the disclosure, the detailed description for known related configurations or functions may be omitted when it is deemed that such description may obscure essential points of the disclosure.

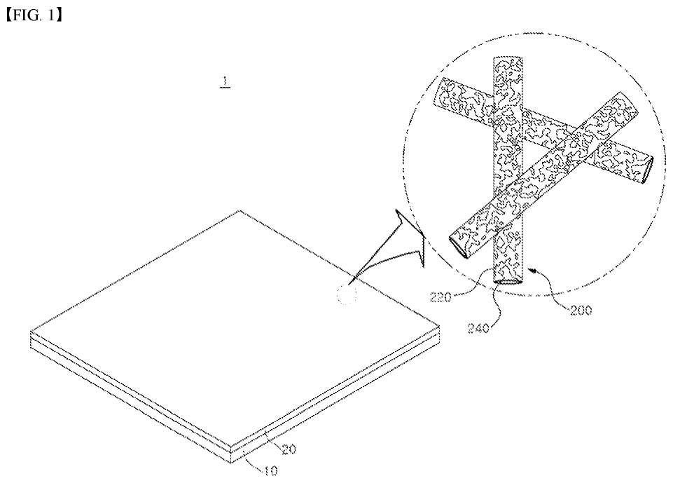

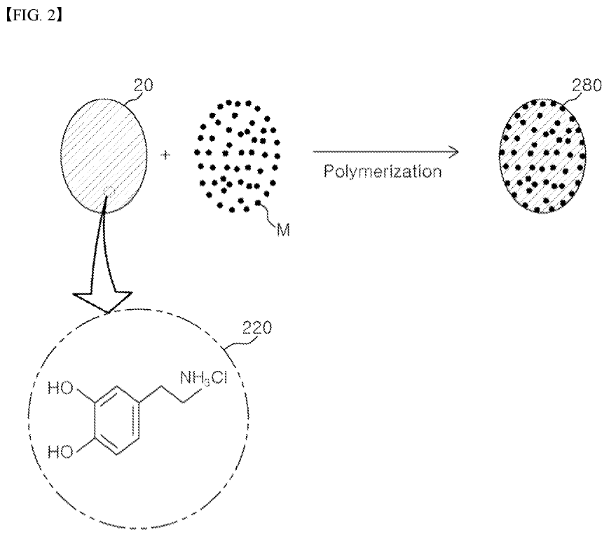

FIG. 1 is a schematic view showing a metal material extraction sheet 1 according to an embodiment of the present disclosure, and FIG. 2 is a diagram conceptually representing a process in which dopamine 220 in the surface layer 20 of FIG. 1 reacts with a metal material M, so that the dopamine 220 is polymerized into polydopamine 280.

Referring to FIGS. 1 and 2, the metal material extraction sheet 1 having a predetermined area may be provided, wherein dopamine 220 whose color is changed when reacting with a metal material is provided on at least a part of the outer surface of the metal material extraction sheet.

For example, the metal material extraction sheet 1 according to an embodiment of the present disclosure may include a sheet layer 10 having a predetermined area, and a surface layer 20 disposed on a side of the sheet layer 10 and provided with the dopamine 220.

In the present embodiment, by way of example, the metal material extraction sheet 1 is described as being provided with the sheet layer 10 and the surface layer 20 separately, and the dopamine 220 being provided in the surface layer 20.

However, the technical idea of the present disclosure is not limited thereto, and the sheet layer 10 may be optionally provided. That is, when the sheet layer 10 is excluded, the surface layer 20 may be understood as the metal material extraction sheet 1. The metal material extraction sheet 1 of this embodiment may remove the metal material M from the user's skin by coming into contact with or being rubbed against it. At this time, the metal material M removed from the user's skin may be combined with the dopamine 220 provided on the surface layer 20, and when the dopamine 220 is combined with the metal material M, the dopamine 220 may be polymerized into the polydopamine 280 at an increased polymerization rate. During the polymerization of dopamine 220 into the polydopamine 280, a color change occurs, and the user can visually grasp the metal material M removed from the skin through such color change. That is, the color of the surface layer 20 of the metal material extraction sheet 1 may change as the surface layer reacts with the metal material M, and the user can grasp the metal material M removed from the skin depending on the color change.

In addition, as the dopamine 220 is polymerized into the polydopamine 280, the adhesion may be increased by the polydopamine 280. Accordingly, the metal material extraction sheet 1 can easily adsorb the metal material M.

The metal material extraction sheet 1 may be manufactured and used as a mask pack, patch, makeup cotton, or the like. However, the present disclosure is not limited thereto, and the metal material extraction sheet 1 is applicable to every place where it is necessary to determine the presence of metal material M.

For example, the metal material extraction sheet 1 may be applied to cleaning products such as a mop, and in this case, it is possible to grasp how much metal material M exists in the house through the change in color of the mop after cleaning.

The sheet layer 10 may be provided in a plate-shaped sheet having a predetermined area, and may be formed of a material that can be deformed by an external force. For example, the sheet layer 10 may be provided as a polymer which is a material for a mask pack used in the cosmetic field. In addition, the thickness of the sheet layer 10 may be approximately between 0.1 mm and 10 mm.

However, as long as the sheet layer 10 has a structure capable of supporting the surface layer 20 to be described below, there may be no limit to the material, shape, and size thereof.

The surface layer 20 may include dopamine 220 capable of being combined with the metal material M. Here, dopamine may be understood as a catecholamine-based organic compound whose chemical formula is C₈H₁₁NO₂.

The dopamine 220 may be polymerized into the polydopamine 280 through a spontaneous oxidation reaction under basic conditions. In this case, it may take a long period of time for the dopamine 220 to be polymerized into the polydopamine 280.

The inventors found that, when the dopamine 220 is combined with the metal material M, the rate at which the dopamine 220 is polymerized into the polydopamine 280 is accelerated, and implemented the metal material extraction sheet 1 by incorporating the dopamine 220 thereinto.

In addition, a change in color may occur during the polymerization of the dopamine 220 to the polydopamine 280. That is, since the surface layer 20 contains the dopamine 220, the color of the surface layer 20 may change by reacting with the metal material M on the user's skin. For example, the surface layer 20 containing the dopamine may be white before the reaction with the metal material M, but may turn brown after the reaction with the metal material M. In this case, the brown color may be implemented by the polydopamine 280 (see FIG. 2).

The metal material M that can react with the dopamine may contain heavy metals and metals. Here, the heavy metal may be understood as any one of elements having a specific gravity greater than 4.5, which is between copper and lead in the periodic table, and has an atomic mass of 63.55 to 200.60. For example, the heavy metals may include iron, manganese, aluminum, beryllium, lead, cadmium, and arsenic. Heavy metals and metals may be mixed with dust in the atmosphere and may adhere to human skin during outdoor activities. In addition, metals such as magnesium may be included in raw materials for cosmetics.

The raw material constituting the surface layer 20 may include the dopamine 220 and the polymer 240. These dopamine 220 and polymer 240 are formed of nanofibers 200 by electrospinning to be described later, and the nanofibers 200 are intertwined or aligned to form the surface layer 20. Here, the use of electrospinning technology has the advantage of being able to easily synthesize a one-dimensional nanofiber membrane having a high surface area, and it will be described later in detail.

In addition, the dopamine 220 and the polymer 240, which are raw materials constituting the surface layer 20, may be provided in a predetermined ratio. For example, the weight ratio of the dopamine 220 and the polymer 240 may be between 1:1 and 1:10. The ratio of the dopamine 220 in the surface layer 20 may be related to the degree to which the color change occurs after being combined with the metal material M. For example, as the ratio of the dopamine 220 in the surface layer 20 is higher, even if a small amount of metal material M is combined with the dopamine, the surface layer 20 may cause a more distinct color change.

The polymer 240 may include a PVP polymer or a PAN polymer, and be used as a mixed polymer containing at least one of polyurethane (PU), nylon, polyvinylidenefluoride (PVdF), polyhydroxybutyrate (PHB), polyetherimide (PEI), polycaprolactone (PCL) and polylactic acid (PLA).

In addition, the raw material constituting the surface layer 20 may further include a functional raw material in addition to the dopamine 220 and the polymer 240. For example, the functional raw material may include one or more of a vitamin, a whitening agent, a skin tone changing agent, an anti-wrinkle agent, an anti-aging agent, an anti-acne agent, a moisturizing agent, and a nutritional agent.

The surface layer 20 may be formed of the nanofibers 200 including the dopamine 220 and the polymer 240. In addition, the surface layer 20 may have a porous membrane structure in which the nanofibers 200 are randomly intertwined or aligned in a predetermined direction and laminated.

In addition, at least one of the sheet layer 10 and the surface layer 20 may have a structure capable of easily absorbing liquid. For example, the nanofibers 200 forming the surface layer 20 may have a diameter of 10 nm to 500 nm, and the average pore size may be 0.1 μm to 1.0 μm. Since the nanofibers 200 have such a diameter and an average size of pores, it can be impregnated with a liquid (for example, a basic liquid having a pH of 8.5 or more), so that the above-described dopamine 220 can be easily polymerized into the polydopamine 280.

In addition, the sheet layer 10 or the surface layer 20 may be formed to exhibit hydrophilicity, so that it can absorb an aqueous solution well.

In addition, the above-described surface layer 20 may be a layer for being brought into direct contact with the user's skin.

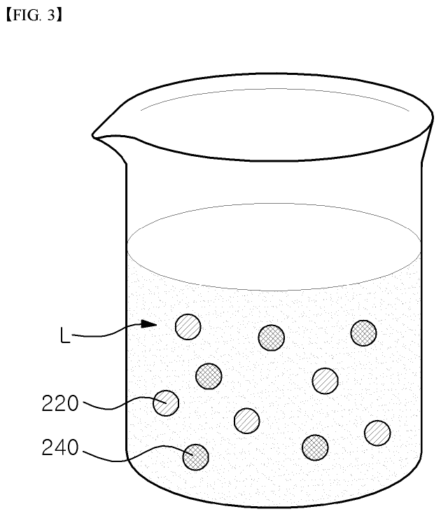

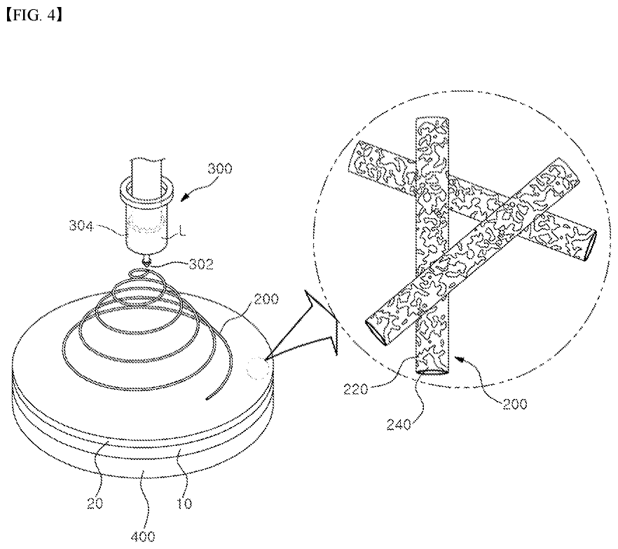

FIG. 3 represents a solution for preparing the surface layer 20 of FIG. 1, and FIG. 4 schematically represents the electrospinning apparatus 300 for preparing the surface layer 20 of FIG. 1 and the metal material extraction sheet 1 prepared by the electrospinning apparatus 300.

Referring to FIGS. 3 and 4, the electrospinning device 300 may include a raw material tank 304 storing a spinning solution L, a control valve (not shown) for controlling the injection amount of the spinning solution L stored in the raw material tank 304, and an injection nozzle 302 for injecting the spinning solution L. In addition, a current collecting plate 400 may be provided for collecting the spinning solution L being subjected to electrospun. Here, the spinning solution L may include the above-described dopamine 220 and polymer 240. In addition, the spinning solution L may further contain a functional raw material (not shown).

A process in which the metal material extraction sheet 1 is prepared using the electrospinning apparatus 300 described above is as follows.

According to an embodiment of the present disclosure, a method of preparing the metal material extraction sheet 1 may include the steps of preparing the spinning solution L containing the dopamine 220 (S1); electrospinning the spinning solution L to form the nanofibers 200 (S2); and laminating the nanofibers 200 on the sheet layer 10 (S3).

Here, the nanofibers 200 may be laminated on the sheet layer 10 and form the above-described surface layer 20.

In addition, the sheet layer 10 may be manufactured through the electrospinning using an electrospinning apparatus 300. However, the present disclosure is not limited thereto, and the sheet layer 10 may be a sheet manufactured by a known method.

The metal material extraction sheet 1 of FIGS. 1 and 2 described above has been described by exemplifying that the surface layer 20 is provided on one surface of the sheet layer 10, but the present disclosure is not limited thereto, and the surface layer 20 may be provided on both surfaces of the sheet layer 10.

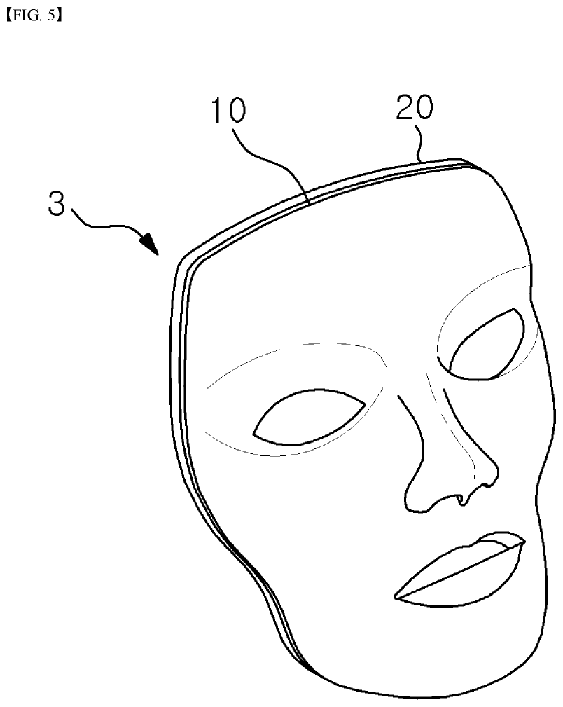

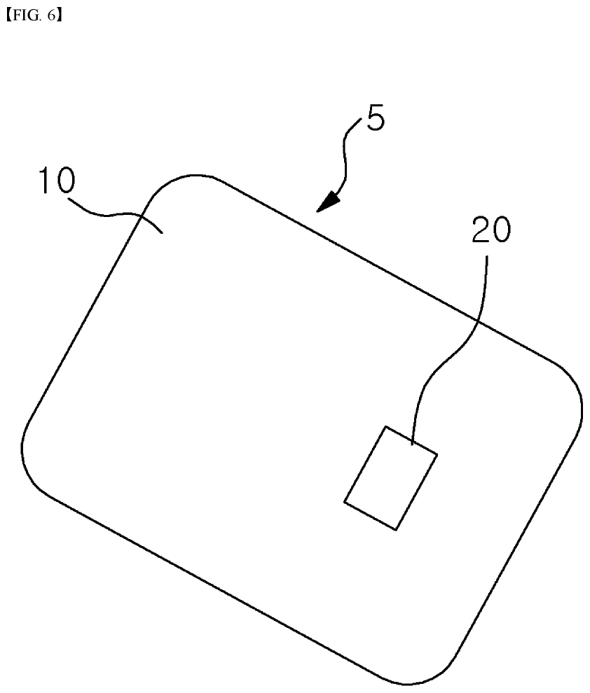

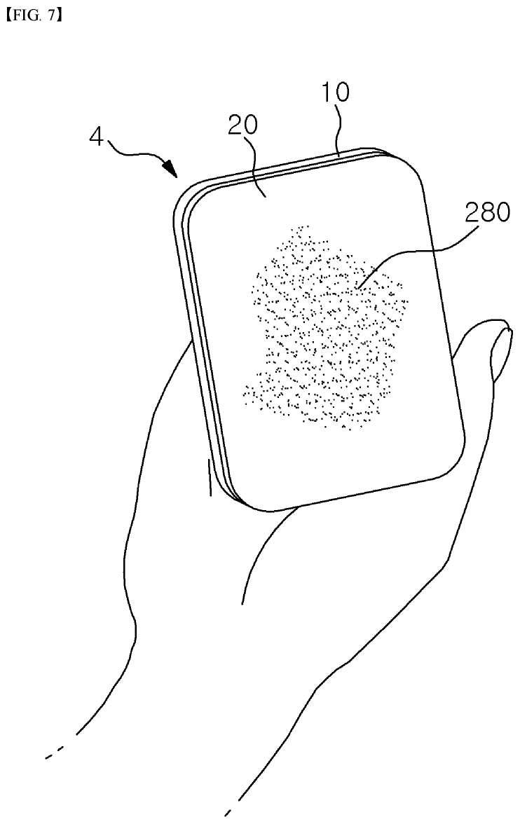

FIG. 5 represents a mask pack 3 to which the metal material extraction sheet 1 of FIG. 1 is applied, FIG. 6 represents a case where the metal material extraction sheet 1 of FIG. 1 is applied to a part of a patch 5, and FIG. 7 represents a case where the metal material extraction sheet 1 of FIG. 1 is applied to a cotton pad 4.

FIGS. 5 to 7 illustrate cases where the above-described metal material extraction sheet 1 is applied as a constitutional component.

Referring to FIG. 5, the metal material extraction sheet 1 may be formed in the shape of a human face and prepared as the mask pack 3. Here, the surface layer 20 may be formed on the inner side of the metal material extraction sheet 1, so that it can be brought into contact with the human skin.

In addition, the metal material extraction sheet 1 may be deformed to correspond to the shape of the user's body (e.g., face), and the surface layer 20 may be provided on the body (face) side.

When this mask pack 3 is attached to and removed from the face, heavy metals or metals on the face may adhere to the surface layer 20 of the mask pack 3, thereby causing a color change of the mask pack 3. Here, the user can grasp the removed amount of heavy metal or metal through the color change of the mask pack 3.

Referring to FIG. 6, the metal material extraction sheet 1 may be prepared as a product that is applied to a part of the patch 5 and detects the metal material M.

The patch 5 may be a skin care device or a medical device configured to be attached to a part of the body, and to detect the metal material M by forming the surface layer 20 including dopamine 220 on a part of the patch 5.

For example, the patch 5 may be a medicated patch for being attached to a part of the body, and detect the metal material M on the skin by forming the surface layer 20 on a part of the medicated patch, while serving as the medicated patch. Here, the surface area of the surface layer 20 may be 1/10 or less of the surface area of the patch 5.

Referring to FIG. 7, the metal material extraction sheet 1 may be applied to a part of a pad 4. FIG. 7 represents a case where the color of the surface layer 20 is changed through the combination of the heavy metals on the skin with the dopamine (220), and the polymerization of the dopamine (220) into the polydopamine 280, which are caused by bringing the pad 4 into contact with or rub the same against the skin. The cosmetic pad 4 may be formed of a conventionally used material. For example, the pad 4 is formed of cotton or a polymer.

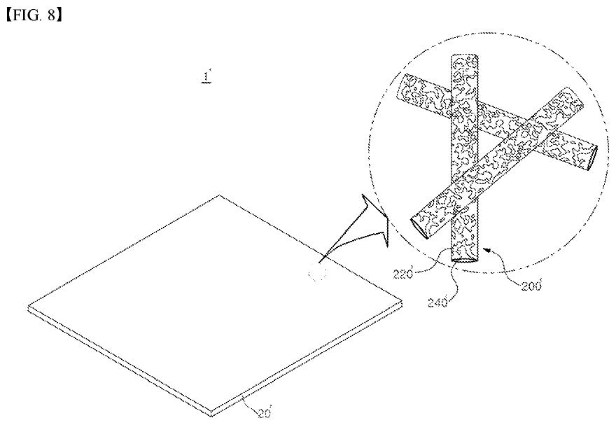

FIG. 8 is a schematic view representing a metal material extraction sheet according to another embodiment of the present disclosure.

Hereinafter, a metal material extraction sheet 1′ according to another embodiment of the present disclosure will be described with reference to FIG. 8. However, since there is a major difference in that the sheet layer 10 is not provided compared to the above-described embodiments, such difference will be primarily described, while for the like elements, the reference is made to the description of the above-described embodiments and the like reference numerals are used. In this embodiment, a surface layer 20′ may be understood as the metal material extraction sheet 1′.

Referring to FIG. 8, the metal material extraction sheet 1′ has a predetermined area, and a dopamine 220′ which reacts with the metal material and changes its color may be provided on a part of the outer surface.

The metal material extraction sheet 1′ is formed by intertwining or aligning in a certain direction nanofibers 200′ in which polymer 240′ and the dopamine 220′ are mixed in a preset ratio. Here, the diameter of the nanofibers 200′ may be between 10 nm and 50 nm, and the average pore size of the nanofibers 200′ may be between 0.1 μm and 1.0 μm.

In addition, a weight ratio of the dopamine 220′ and the polymer 240′, which are raw materials of the metal material extraction sheet 1′, may be between 1:1 and 1:10.

In addition, although not shown in the drawings, the metal material extraction sheet 1′ may further include a functional raw material. Here, the functional raw material may include one or more of a vitamin, a whitening agent, a skin tone changing agent, an anti-wrinkle agent, an anti-aging agent, an anti-acne agent, a moisturizing agent, and a nutritional agent.

In addition, the metal material extraction sheet 1′ may be impregnated with a liquid. Here, the liquid may be a basic material having a pH of 8.5 or higher.

The metal material extraction sheet 1′ illustrated in FIG. 8 may have the same configuration as the surface layer 20 illustrated in FIG. 1, so detailed descriptions thereof will be omitted.

Further, a method of preparing the metal material extraction sheet 1′ may include the steps of preparing the spinning solution L containing the dopamine 220′ (S1′); electrospinning the spinning solution L to form the nanofibers 200′ (S2′); and laminating the nanofibers 200′ on a current collecting plate (S3′). Here, the spinning solution may include the above-described dopamine 220′ and polymer 240′. In addition, the spinning solution may further contain a functional raw material (not shown).

Hereinafter, the action and effect of the above-described metal material extraction sheet will be described.

The metal material extraction sheet 1, 1′ may remove the metal material M from the user's skin by coming into contact with or being rubbed against it. At this time, the metal material M removed from the user's skin may be combined with the dopamine 220, 220′, and when the dopamine (220, 220′) is combined with the metal material M, the dopamine 220, 220′ may be polymerized into the polydopamine 280 at an increased polymerization rate. During the polymerization of dopamine 220 into the polydopamine 280, a color change occurs, and the user can visually grasp the metal material M removed from the skin through such color change.

That is, the color of the dopamine 220, 220′ of the metal material extraction sheet 1, 1′ may change as it reacts with the metal material M, and the user can grasp the metal material M removed from the skin through the color change.

In addition, as the dopamine 220, 220′ is polymerized into the polydopamine 280, the adhesion may be increased by the polydopamine 280. In this case, the metal material M can be more easily removed by the metal material extraction sheets 1, 1′.

In addition, the dopamine 220, 220′ and the polymers 240, 240′ may be formed into the nanofibers 200, 200′ by the electrospinning. As described above, there is an advantage that a one-dimensional nanofiber membrane having a high surface area can be easily synthesized by using the electrospinning technique.

Hereinafter, embodiments of above-described metal material extraction sheet and a method of preparing a metal material extraction sheet will be listed.

Item 1: A metal material extraction sheet that has a predetermined area, and dopamine whose color changes when reacting with metal material and which is provided on at least a part of an outer surface of the metal material extraction sheet.

Item 2: A metal material extraction sheet of Item 1, wherein the metal material extraction sheet is formed by intertwining or aligning in a predetermined direction nanofibers in which a polymer and the dopamine are mixed in a preset ratio.

Item 3: A metal material extraction sheet of Item 1 and Item 2, wherein the diameter of the nanofibers is between 10 nm and 50 nm.

Item 4: A metal material extraction sheet of Item 1 to Item 3, wherein the average pore size of the nanofibers is between 0.1 μm and 1.0 μm.

Item 5: A metal material extraction sheet of Item 1 to Item 4, wherein the weight ratio of the dopamine and the polymer is between 1:1 and 1:10.

Item 6: A metal material extraction sheet of Item 1 to Item 5, wherein the metal material extraction sheet further comprises a functional raw material, and wherein the functional raw material includes one or more of a vitamin, a whitening agent, a skin tone changing agent, an anti-wrinkle agent, an anti-aging agent, an anti-acne agent, a moisturizing agent, and a nutritional agent.

Item 7: A metal material extraction sheet of Item 1 to Item 6, wherein the metal material extraction sheet is impregnated with liquid.

Item 8: A metal material extraction sheet of Item 1 to Item 7, wherein the liquid is a basic material having a pH of 8.5 or higher.

Item 9: A metal material extraction sheet of Item 1 to Item 8, wherein the metal material extraction sheet further includes a sheet layer having a predetermined area, and the dopamine is provided on one surface of the sheet layer to form a surface layer thereof.

Item 10: A metal material extraction sheet of Item 1 to Item 9, wherein the surface layer is formed in a region of 1/10 or less of the surface area of the sheet layer.

Item 11: A metal material extraction sheet of Item 1 to Item 10, wherein the surface layer is provided on both surfaces of the sheet layer.

Item 12: A mask pack comprising a metal material extraction sheet of Item 1 to Item 11, wherein the metal material extraction sheet is deformable to correspond to a user's body shape, and the dopamine is provided to a user's body side of the metal extraction sheet.

Item 13: A method of preparing a metal material extraction sheet according to one of Item 1 to Item 12, the method comprising: preparing a spinning solution containing dopamine; electrospinning the spinning solution to form nanofibers; and laminating the nanofibers to form the metal material extraction sheet. The metal material extraction sheet according to the embodiment of the present invention, the mask pack including the same, and the method of preparing the metal material extraction sheet have been described as specific embodiments, but these are only examples, and the present disclosure is not limited thereto. The present disclosure should be construed as having the widest scope in accordance with the basic technical ideas disclosed herein. By combining or replacing a part or parts of embodiments disclosed herein, the ordinary skilled in the art may carry out a pattern of a shape which is not explicitly described herein, and however, it should be noted that it shall not depart from the scope of the disclosure. Besides, the ordinary skilled in the art may easily change or modify embodiments disclosed herein based on the disclosure, and however, it is obvious that such change or modification also falls within the scope of the disclosure.

### REFERENCE SIGN LIST

1, 1′: metal material extraction sheet
10: sheet layer
20, 20′: surface layer
220, 220′: dopamine
240, 240′: polymer

1. A metal material extraction sheet that has a predetermined area, and dopamine whose color changes when reacting with metal material and which is provided on at least a part of an outer surface of the metal material extraction sheet.

2. The metal material extraction sheet of claim 1, wherein the metal material extraction sheet is formed by intertwining or aligning in a predetermined direction nanofibers in which a polymer and the dopamine are mixed in a preset ratio.

3. The metal material extraction sheet of claim 2, wherein the diameter of the nanofibers is between 10 nm and 50 nm.

4. The metal material extraction sheet of claim 2, wherein the average pore size of the nanofibers is between 0.1 μm and 1.0 μm.

5. The metal material extraction sheet of claim 2, wherein the weight ratio of the dopamine and the polymer is between 1:1 and 1:10.

6. The metal material extraction sheet of claim 1, wherein the metal material extraction sheet further comprises a functional raw material, and
wherein the functional raw material includes one or more of a vitamin, a whitening agent, a skin tone changing agent, an anti-wrinkle agent, an anti-aging agent, an anti-acne agent, a moisturizing agent, and a nutritional agent.

7. The metal material extraction sheet of claim 1, wherein the metal material extraction sheet is impregnated with liquid.

8. The metal material extraction sheet of claim 7, wherein the liquid is a basic material having a pH of 8.5 or higher.

9. The metal material extraction sheet of claim 1, wherein the metal material extraction sheet further includes a sheet layer having a predetermined area, and the dopamine is provided on one surface of the sheet layer to form a surface layer thereof.

10. The metal material extraction sheet of claim 9, wherein the surface layer is formed in a region of 1/10 or less of the surface area of the sheet layer.

11. The metal material extraction sheet of claim 9, wherein the surface layer is provided on both surfaces of the sheet layer.

12. A sheet mask comprising a metal material extraction sheet according to claim 1, wherein the metal material extraction sheet is deformable to correspond to a user's body shape, and the dopamine is provided to a user's body side of the metal extraction sheet.

13. A method of preparing a metal material extraction sheet, the method comprising:
preparing a spinning solution containing dopamine;
electrospinning the spinning solution to form nanofibers; and
laminating the nanofibers to form the metal material extraction sheet.
